SEQUENCE LISTING (30 sequences)

SEQ ID NO: 1 - protein, 19 aa, Epstein-Barr virus
RDGVRRPQKRPSCIGCKGT

SEQ ID NO: 2 - protein, 26 aa, Epstein-Barr virus
RPFFHPVGEADYFEYHQEGGPDGEPD

SEQ ID NO: 3 - protein, 22 aa, Epstein-Barr virus
DGGRRKKGGWFGKHRGQGGSNP

SEQ ID NO: 4 - protein, 26 aa, Epstein-Barr virus
NPKFENIAEGLRALLARSHVERTTDE

SEQ ID NO: 5 - protein, 26 aa, Epstein-Barr virus
KTSLYNLRRGTALAIPQCRLTPLSRL

SEQ ID NO: 6 - protein, 24 aa, Epstein-Barr virus
PQCRLTPLSRLPFGMAPGPGPQPG

SEQ ID NO: 7 - protein, 22 aa, Epstein-Barr virus
MVFLQTHIFAEVLDAIKDLVMT

SEQ ID NO: 8 - protein, 24 aa, Epstein-Barr virus
FAEVLKDAIKDLVMTKPAPTCNIR

SEQ ID NO: 9 - protein, 19 aa, Epstein-Barr virus
LPPWFPPMVEGAAAEGDDG

SEQ ID NO: 10 - protein, 15 aa, Epstein-Barr virus
EHDLERGPPGPRRPP

SEQ ID NO: 11 - protein, 16 aa, Epstein-Barr virus
IIFIFRRDLLCPLGAL

SEQ ID NO: 12 - protein, 16 aa, Epstein-Barr virus
ALWNLHGQALFLGIVL

SEQ ID NO: 13 - protein, 17 aa, Epstein-Barr virus
AILIWMYYHGPRHSDEH

SEQ ID NO: 14 - protein, 15 aa, Epstein-Barr virus
HDPLPHSPSDSAGND

SEQ ID NO: 15 - protein, 27 aa, Epstein-Barr virus
SGHESDSNSNEGRHHLLVSGAGDGPPL

SEQ ID NO: 16 - protein, 31 aa, Epstein-Barr virus
DPHLPTLLLGTSGSGGDDDDPHGPVQLSYYD

SEQ ID NO: 17 - protein, 20 aa, Epstein-Barr virus
MGSLEMVPMGAGPPSPGGDP

SEQ ID NO: 18 - protein, 25 aa, Epstein-Barr virus
EDPYWGNGDRHSDYQPLGTQDQSLY

SEQ ID NO: 19 - protein, 15 aa, Epstein-Barr virus
ASSYAAAQRKLLTPV

SEQ ID NO: 20 - protein, 21 aa, Epstein-Barr virus
VLVMLVLLILAYRRRWRRLTV

SEQ ID NO: 21 - protein, 17 aa, Epstein-Barr virus
GGSILQTNFKSLSSTEF

SEQ ID NO: 22 - protein, 18 aa, Epstein-Barr virus
LTEWGSGNRTYGPVFMCL

SEQ ID NO: 23 - protein, 13 aa, Artificial
  Synthetic peptide
PKYVKQNTLKLAT

SEQ ID NO: 24 - protein, 15 aa, Artificial
  Synthetic peptide
EAEQLRAYLDGTGVE

SEQ ID NO: 25 - protein, 15 aa, Artificial
  Synthetic peptide
AKTIAYDEEARRGLE

SEQ ID NO: 26 - protein, 13 aa, Artificial
  Synthetic peptide
AAYAAAKAAALAA

SEQ ID NO: 27 - protein, 16 aa, Artificial
  Synthetic peptide
TERVRLVTRHIYNREE

SEQ ID NO: 28 - protein, 20 aa, Artificial
  Synthetic peptide
ESWGAVWRIDTPDKLTGPFT

SEQ ID NO: 29 - protein, 14 aa, Artificial
  Synthetic peptide
AGDLLAIETDKATI

SEQ ID NO: 30 - protein, 17 aa, Artificial [sequence omitted]

(19) United States
(12) Patent Application Publication
(10) Pub. No.: US 2009/0130134 A1
(43) Pub. Date: May 21, 2009

(54) T CD4+ Epitopes of Type I and II Latency Antigens of the Epstein-Barr Virus, Which Can Be Recognized by the majority of individuals in the caucasian populations and applications thereof

(76) Inventors: Veronique Pancre, Orchies (FR); Claude Auriault, Nomain (FR); Stephane Depil, Lille (FR); Bernard Maillere, Versailles (FR); Olivier Morales, Lille (FR); Gaetan Munier, Orsay (FR)

Correspondence Address: MORGAN LEWIS & BOCKIUS LLP, 1111 PENNSYLVANIA AVENUE NW, WASHINGTON, DC, 20004, US

(21) Appl. No.: 11/883,814

(22) PCT Filed: Feb. 2, 2006
(86) PCT No.: PCT/FR06/00229

§ 371 Date: May 7, 2008

(30) Foreign Application Priority Data
Feb. 7, 2005 (FR) 0501198

(51) Int. Cl. A61K 39/245; C07K 14/00; C07K 7/00; C12N 15/11; C12Q 1/70; C12N 15/00; C12N 5/06

(52) U.S. Cl. 424/186.1; 530/324; 530/325; 530/326; 536/23.72; 435/320.1; 435/325; 424/184.1; 435/5

(57) ABSTRACT

The invention relates to immunogenic peptides from EBV type I and II latency antigens, comprising at least one T CD4+ epitope which can be recognized by the majority of individuals in the Caucasian population. The invention also relates to the diagnostic and therapeutic applications of same.

The present invention relates to immunogenic peptides derived from EBV type I and II latency antigens comprising at least one CD4+ T epitope which can be recognized by the majority of individuals in the Caucasian population, and to the diagnostic and therapeutic applications thereof.

The Epstein-Barr virus (EBV) is an oncogenic herpes virus with B lymphocyte tropism, which infects 95% of the adult population. After the primary infection, which is generally asymptomatic in children and sometimes responsible for infectious mononucleosis, in particular in adolescents, EBV persists in the latent state in the B lymphocytes throughout the individual's life. Although, in the majority of cases, EBV infection is effectively controlled by the immune system (normal carriers), in certain cases, it is associated with various tumoral pathologies which can be distinguished by the expression of the latency antigens.

Type I latency corresponds to the isolated expression of the EBNA1 antigen. This expression profile is found in endemic-type Burkitt's lymphomas, encountered mainly in Africa and in New Guinea (unlike the sporadic form, which is not generally related to EBV). This type of latency is characterized by the weak recognition of the tumor cells by cytotoxic CD8+ T lymphocytes. This weak recognition capacity is explained by: (i) the absence of EBNA1-derived epitopes presented at the level of the class I MHC (repeats of glycine-alanine sequences preventing degradation by the proteasome); (ii) the decrease in adhesion molecules; (iii) the decrease in expression of the TAP 1 and 2 proteins; (iv) the low expression of class I HLA molecules (or HLA I). The tumor cells can nevertheless be recognized by cytotoxic CD4+ T lymphocytes, since they exhibit a normal expression of class II HLA (or HLA II) molecules, associated with normal processing.

In type II latency, an expression of latency antigens limited to EBNA1, LMP1 and LMP2 is found. This type of latency is encountered in:
undifferentiated nasopharyngeal carcinoma (NPC) observed mainly in Southern China and in North Africa;
40 to 50% of Hodgkin's diseases. The association with EBV is more common in the mixed cellularity forms (50-75%) than in the scleronodular forms (15-30%);
certain T and NK lymphomas, in particular naso-sinusoidal T/NK lymphomas.

As regards Hodgkin's disease, a normal expression of class I HLA molecules and of TAP 1 and 2 transporters, and also a considerable expression of class II HLA molecules, by Reed-Steinberg cells is observed. However, type II latency is characterized by the absence of immunodominant epitopes (EBNA 3A, 3B, 3C). In fact, while there exists a CTL response directed against the LMP1 and LMP2 proteins, the latter remains weak. It therefore appears to be advantageous to stimulate this response in the context of immunotherapy protocols.

Type III latency, which corresponds to the expression of all the latency proteins (EBNA1, EBNA2, EBNA3A-C, EBNA-LP, LMP1 and LMP2), is observed in post-transplantation lymphomas, related to a state of severe immunodepression.

The control of latent EBV infection by EBV-specific T lymphocytes is essential (Rooney et al., Lancet, 1995, 345, 913; Munz, J. Exp. Med., 2000, 191, 1649-1660; Leen et al., Virol., 2001, 75, 8649-8659). It is intended to prevent an uncontrolled proliferation of EBV-infected cells, which is observed in immuno-depressed individuals (post-transplantation proliferative syndromes, type III latency).

Schematically, the EBNA3A-C and LMP2 proteins are the main targets for CD8+ T lymphocytes, whereas EBNA1 and LMP1 are mainly recognized—even more exclusively for EBNA1—by CD4+ T lymphocytes. Consequently, the immune control of tumors associated with EBV type I and II latencies is based to a large extent on the cytotoxic CD4+ T lymphocytes capable of recognizing and lysing the target cells expressing EBNA1 and/or LMP1 epitopes, presented by HLA II molecules. Furthermore, the CD4+ T lymphocytes are necessary for maintaining, in vivo, a cytotoxic response mediated by CD8+ T lymphocytes capable of recognizing and lysing the target cells expressing LMP2 epitopes.

No immunotherapy protocol for tumoral pathologies associated with a type I or type II latency, which is really effective and easy to implement, currently exists. In fact, cell immunotherapy, which is based on the preparation of polyclonal cytotoxic T lymphocytes obtained by in vitro stimulation of T lymphocytes with autologous B lymphoblastoid lines transformed with EBV (LCL), is lengthy to implement (LCLs obtained in 4 to 6 weeks) and not very effective insofar as the degree of expansion of the EBV-specific T lymphocytes is low and requires the addition of a mitogenic cocktail in patients suffering from Hodgkin's disease; in addition, no complete tumor remission is observed.

These observations support the use of antigenic peptides specific for the EBV latency antigens EBNA1, LMP1 and LMP2 and capable of stimulating a strong CD4+ T response, in immunotherapy (immunization or cell therapy), for the prevention and treatment of tumoral pathologies associated with type I or type II latencies.

Such peptides, which are recognized by EBV-specific CD4+ T cells, are also useful as reagents in a diagnostic test for EBV infection or for associated tumoral pathologies, or for monitoring the treatment in humans, based on the direct detecting (lymphocyte proliferation assay) or indirect detection (production of antibodies, of cytokines, etc.) of said CD4+ T cells.

CD4+ T lymphocytes or CD4+ T cells are activated under the effect of the presentation of antigenic peptides by the molecules of the type II major histocompatibility complex borne by antigen-presenting cells; in humans, they are called HLA II molecules, for Human Leucocyte Antigen type II. These antigenic peptides, called T epitopes, result from the proteolytic degradation of the antigens by the antigen-presenting cells. They have variable lengths, generally from 13 to 25 amino acids, and possess a sequence which makes them capable of binding to the HLA II molecules. It is well known that, in the same way as the native antigen, a peptide comprising a CD4+ T epitope is capable of stimulating, in vitro, CD4+ T cells which are specific for said epitope or of recruiting them in vivo. It is therefore sufficient to produce a CD4+ T response.

However, one of the major problems which limits the use of these peptides as an antigen is the identification of the CD4+ T epitopes, given that their sequence varies from one individual to the other due to the polymorphism of the HLA II molecules. In fact, HLA II molecules are heterodimers consisting of an alpha (α) chain and a beta (β) chain which are polymorphic. Four types of HLA II molecules exist per individual (2 HLA-DR, 1 HLA-DQ and 1-HLA-DP), named according to the allele encoding the beta chain which is the most polymorphic. The HLA-DR molecule is highly polymorphic. In fact, although its alpha chain has only 3 alleles, the beta (β) chain encoded by the DRB1 gene, which is the most widely expressed, has, to date, 458 alleles. For the HLA-DQ and HLA-DP molecules, the two chains (α and β) of which they are formed are polymorphic, but they have fewer alleles. 8 DQA1 alleles (a chain of HLA-DQ), 56 DQB1 alleles (βchain of HLA-DQ), 20 DPA1 alleles (a chain of HLA-DP) and 110 DPB1 alleles (β chain of HLA-DP) have been counted. However, the combination between the two α and β chains encoded by these alleles gives rise to numerous HLA-DQ and HLA-DP molecules. Due to this polymorphism, these isoforms have different binding properties with respect to one another, which implies that they can bind different peptides of the same antigen. Thus, each individual recognizes, in an antigen, a collection of peptides whose nature depends on the HLA II molecules which characterize it. Since there exists a large number of HLA II alleles, there therefore exists, in a given sequence, a considerable repertoire of T epitopes of very different sequences, each specific for a different allele.

Thus, a peptide capable of stimulating an EBV-specific CD4+ T response in some individuals may be inactive in the majority of other individuals, because the latter do not recognize the EBV antigens via the same epitopes.

Most of the CD4+ T epitopes of the EBNA1, LMP1 and LMP2 antigens which are known, have a defined restriction for a single HLA DR, DQ or DP molecule (table I).

TABLE I
CD4+ T epitopes of the EBNA1, LMP1 and LMP2 antigens

| EBV | CD4+ T | | |
|---|---|---|---|
| antigen | epitope | HLA II restriction | Reference |
| EBNAI | 475-489 | DR11 | Leen et al. J. Virol., |
| | | | 2001, 75, 8649-8659 |
| | 475-489 | DP | Taylor et al., J. |
| | | | Virol., 2004, 78, 768-778 |
| | 481-500 | DQ | Paludan et al., J. |
| | | | Immunol., 2002, 169, |
| | | | 1593-1603 |
| | 482-496 | DR4, DR7 | Kruger et al., J. |
| | | DR11, DR15 | Immunother., 2003, 26, |
| | | | 212-221 |
| | 485-499 | [DR11, DR13, DR15, | Leen et al., mentioned |
| | | DQ1, DQ3, DQ6]* | above |
| | 514-527 | DR1 | Paludan et al., |
| | | | mentioned above |
| | 514-533 | DR1* | Leen et al., mentioned |
| | | | above |
| | 515-527 | DR1 | Khanna et al., |
| | | | Virology, 1995, 214, |
| | | | 633-637 |
| | 515-528 | DR1 | Leen et al., mentioned |
| | | | above |
| | | | Taylor et al., |
| | | | mentioned above |
| | 518-530 | DP3/DR4 | Voo et al., Cancer |
| | | | Res., 2002, 62, 7195- |
| | | | 7199 & US application |
| | | | 2004/0141995 |
| | 519-533 | DR4, DQ3* | Leen et al., mentioned |
| | | | above |
| | 529-543 | DR14 | Taylor et al., |
| | | | mentioned above |
| | 529-543 | [DR13, DQ3, DQ5, | Leen et al., mentioned |
| | | DQ6]* | above |
| LMP1 | 66-85** | Not determined | Application |
| | | | WO 2004/001424 |
| | 130-144 | [DR1, DR4, DQ1, | Leen et al., mentioned |
| | | DQ3]* | above |
| | 208-224 | Not determined | Application WO |
| | | | 2004/041849 |
| | 212-226 | DQ2* | Leen et al., mentioned |
| | | | above |
| | 340-354 | Not determined | Leen et al., mentioned |
| | | | above |
| LMP2 | 149-163 | [DR1, DR13, DQ5]* | Leen et al., mentioned |
| | | | above |
| | 169-182 | [DR1, DR13, DQ5, | Leen et al., mentioned |
| | | DQ6]* | above |
| | 224-243 | Not determined | Leen et al., mentioned |
| | | | above |
| | 369-388** | Not determined | Application |
| | | | WO 2004/01424 |
| | 385-398 | Not determined | Leen et al., mentioned |
| | | | above |

*Potential restriction element determined according to the HLA II haplotype of responder individuals.
**Use for inducing immune tolerance for the purpose of treating autoimmune diseases.

Consequently, such peptides will not generally be recognized by all the predominant HLA II molecules in the Caucasian population. In fact, for the HLA-DR molecules, for example, about 10 or so alleles are necessary in order to cover more than 60% of the gene frequency found in the Caucasian population and therefore to relate to more than 85% of this population.

The inventors have identified CD4+ T epitopes derived from the EBNA1, LMP1 and LMP2 latency antigens of EBV, which epitopes can be recognized by the HLA II molecules predominant in the Caucasian population and are therefore capable of inducing an EBV-specific CD4+ T response in the majority of individuals in this population who receive an immunization or a cell therapy using peptides including these epitopes.

Consequently, a subject of the present invention is an immunogenic peptide of the Epstein-Barr virus (EBV), comprising at least one CD4+ T epitope of one of the EBNA1, LMP1 or LMP2 latency antigens, which can be presented by at least 7 different HLA II molecules chosen from the HLA-DR1, HLA-DR3, HLA-DR4, HLA-DR7, HLA-DR11, DLA-DR13, HLA-DR15, HLA-DRB3, HLA-DRB4, HLA-DRB5 and HLA-DP4 molecules, which peptide is selected from the group consisting of:

a) the peptides of, respectively, 26, 26 and 24 amino acids whose sequence extends from positions 475 to 500, 514 to 539, and 529 to 552 of EBNA1,
b) the peptide of 16 amino acids whose sequence extends from positions 68 to 83 of LMP1,
c) the peptides of, respectively, 21 and 17 amino acids whose sequence extends from positions 224 to 244 and 372 to 388 of LMP2, and
d) the variants of the same length and defined by the same positions as the peptides defined in a), b) and c), which variants are obtained by substitution of at least one amino acid of said peptides defined in a), b) and c) with another amino acid, and exhibit a binding activity of less than 1000 nM, with respect to at least 7 different HLA II molecules chosen from HLA-DR1, HLA-DR3, HLA-DR4, HLA-DR7, HLA-DR11, HLA-DR13, HLA-DR15, HLA-DRB3, HLA-DRB4, HLA-DRB5 and HLA-DP4.

The peptides according to the invention, whose sequences are those specified above, have properties which are distinct from the peptides of the prior art, and in particular the following properties:
HLA II-molecule-binding activity: The peptides according to the invention have a high affinity for the majority of the HLA II molecules predominant in the Caucasian population and are therefore capable of being presented by HLA II molecules in the majority of individuals in the Caucasian population. These peptides have a binding activity<1000 nM for at least 7 of the HLA II molecules chosen from HLA-DR1, HLA-DR3, HLA-DR4, HLA-DR7, HLA-DR11, HLA-DR13, HLA-DR15, HLA-DRB3, HLA-DRB4, HLA-DRB5 and HLA-DP4, which correspond to the HLA II molecules for which the allelic frequency is greater than 5% in the Caucasian population. The binding activity can be measured by means of an HLA II/peptide competition binding assay, with immunoenzymatic revelation, according to the principle described in U.S. Pat. No. 6,649,169 for HLA-DR molecules, and in PCT international application WO 03/040299 for HLA-DP4 molecules, and also in the Articles under the names of Texier et al., J. Immunol., 2000, 164, 3177 and Eur. J. Immunol, 2001, 31, 1837, and Castelli et al., J. Immunol., 2002, 169, 6928-6934.
Immunogenicity: The peptides according to the invention are recognized by CD4+ T lymphocytes in the majority of individuals in the Caucasian population and contain T epitopes naturally presented during EBV infection. Consequently, these peptides are therefore capable of inducing EBV-specific CD4+ T cells from the precursors present in the majority of naïve individuals, or else of stimulating such cells in the majority of individuals seropositive for EBV. The immunogenicity of the peptides can be determined, in particular from peripheral blood mononuclear cells (PBMCs), by means of any appropriate assay known to those skilled in the art, for instance: a cell proliferation assay, an ELISPOT test (assaying of cytokine-producing cells) or a test for assaying cytokines (IFN-γ, IL-2, IL-4 and IL-10).
Cytotoxic activity: Th1-type CD4+ T lymphocyte clones are isolated from T lymphocytes generated by in vitro stimulation of PBMCs, using the peptides according to the invention. The clones thus obtained are placed in the presence of EBV-positive targets with the same HLA specificity: lymphocytes derived from the same individual and transformed with EBV (LCLs) or EBV-positive tumor lines (Hodgkin's, T-NK lymphomas) in the case of HLA identity. The presence of cytotoxic CD4+ T lymphocytes specific for the EBV type I or II latency antigens, capable of lysing the EBV-positive target cells with the same specificity, is analyzed by means of a conventional cytotoxicity assay, in particular by measuring ⁵¹Cr release.

The invention encompasses the peptides whose sequence corresponds to that of a fragment of the EBNA1, LMP1 or LMP2 protein of any EBV isolate, including the natural variants isolated from EBV-infected individuals. The sequences of the genome and of the corresponding proteins of the various EBV isolates are available in the databanks, in particular in that of the NCBI (http://www.ncbi.nlm.nih.gov).

The positions of the peptides are indicated relative the sequences having the following accession numbers in the NCBI database: NP_{—}039875 (EBNA1), CAA26023 (LMP1) and AAA45887 (LMP2). For example, the peptide of 26 amino acids which extends from positions 475 to 500 of ENBA1, extends from the asparagine (N) residue at position 475 to the glutamic acid (E) residue at position 500 of the NCBI sequence NP_{—}039875; it is in particular represented by the sequence NPKFENIAEGLRALLARSHVERTTDE (SEQ ID NO: 4).

Based on these indications, those skilled in the art are able to readily determine the corresponding positions in the proteins of other EBV isolates, which can vary by a few amino acids due to short insertions and/or deletions in the amino acid sequence of said proteins.

The invention also encompasses the variants obtained from the above sequences by substitution of one or more amino acids in the sequence of the peptide, provided that said variant conserves a high affinity (binding activity<1000 nM) for at least 7 of the HLA II molecules predominant in the Caucasian population, as defined above, and that it is immunogenic. The amino acid residues involved in the binding to the HLA-DR and HLA-DP molecules (anchoring residues) and the effect of modifications of these residues on the binding to said molecules (affinity, specificity) are known to those skilled in the art; U.S. Pat. No. 6,649,166 describes the method of binding of peptides to HLA DR molecules, including the determination of the anchoring residues P1, P4, P6, P7 and P9 and the effect of mutations of these residues on the affinity and the specificity of HLA DR molecule binding. PCT international application WO 03/040299 teaches that the binding to HLA-DP4 involves the residues at P6, P1 and/or P9, which should be almost exclusively aromatic or hydrophobic, whereas the residue at P4 can be any amino acid residue.

The invention also encompasses the modified peptides derived from the above peptides by the introduction of any modification at the level of one or more amino acid residue(s), of the peptide binding or of the ends of the peptides, provided that said modified peptide conserves a high affinity (binding activity<1000 nM) for at least 7 of the HLA II molecules predominant in the Caucasian population, as defined above, and that it is immunogenic. These modifications, which are introduced into the peptides by conventional methods known to those skilled in the art, include, in a nonlimiting manner: the substitution of an amino acid with a nonproteinogenic amino acid (D amino acid or amino acid analogue); the addition of chemical groups (lipid, oligosaccharide or polysaccharide) to a reactive function, in particular the R side chain; the modification of the peptide binding (—CO—NH—), in particular by means of a binding of retro or retroinverso type (—NH—CO—) or a binding different from the peptide binding; cyclization; the fusion of a peptide (epitope of interest for immunization; tag useful for purification of the peptide, in particular in a form cleavable by a protease); the fusion of the sequence of said peptide with that of a protein, in particular an α or β chain of an HLA II molecule or the extracellular domain of said chain; coupling to an appropriate molecule, in particular a label, for example a fluorochrome. These modifications are intended, in particular, to increase the stability, the solubility or the immunogenicity or to facilitate the purification or the detection, either of the peptide according to the invention, or of CD4+ cells specific for said peptide.

According to an advantageous embodiment of said peptide, said HLA II molecule comprises a β chain encoded, respectively, by the alleles DRB1*0101 (HLA-DR1 molecule), DRB1*0301 (HLA-DR3 molecule), DRB1*0401 (HLA-DR4 molecule), DRB1*0701 (HLA-DR7 molecule), DRB1*1101 (HLA-DR11 molecule), DRB1*1301 (HLA-DR13 molecule), DRB1*1501 (HLA-DR15 molecule), DRB3*0101 (HLA-DRB3 molecule), DRB5*0101 (HLA-DRB5 molecule), DP*0401 or DP*0402 (HLA-DP4 molecule).

According to an advantageous arrangement of this embodiment, said peptide is selected from the group consisting of the sequences SEQ ID NOS: 4, 5, 6, 11, 20 and 21.

The subject of the present invention is also a polyepitope fragment, characterized in that it comprises at least two identical or different epitopes of EBV, including at least one CD4+ T epitope whose sequence is that of a peptide as defined above.

For the purpose of the present invention, the term “polyepitope fragment” is intended to mean an artificial or synthetic sequence obtained from fragments of one or more EBV antigens, which sequence does not correspond to any sequence naturally present in an EBV antigen.

Preferably, said polyepitope fragment is from 20 to 1000 amino acids in length, preferably from 20 to 100 amino acids.

Said polyepitope fragment advantageously comprises a tag fused to one of its ends, for the purification or detection of said fragment. The tag, in particular a polyhistidine sequence or a B epitope of another virus, is preferably separated from the polyepitope sequence by a cleavage site for a protease in such a way as to isolate the polypeptide sequence from the fusion.

According to an advantageous embodiment of said polyepitope fragment, it comprises a CD4+ T epitope whose sequence is that of a peptide as defined above and at least one other epitope selected from the group consisting of:
a CD8+ T epitope of an EBV protein presented by an HLA I molecule and recognized by EBV-specific cytotoxic T lymphocytes; the sequences of the EBV CD8+ T epitopes are known to those skilled in the art and include, in particular, the peptides 51-60, 125-133, 156-164 and 159-167 of LMP1 and the peptides 131-139, 200-208, 236-244, 340-350, 419-427, 426-434, 442-451, 447-455 and 453-461 of LMP2;
a B epitope of an EBV antigen recognized specifically by antibodies directed against EBV, and
a natural or synthetic universal CD4+ T epitope such as the tetanus toxin TT peptide 830-846 (O'Sullivan et al., J. Immunol., 1991, 147, 2663-2669), the flu virus hemagglutinin HA peptide 307-319 (O'Sullivan et al., mentioned above), the PADRE peptide (KXVAAWTLKAA; Alexander et al., Immunity, 1994, 1, 751-761), peptide 45-69 of the HIV-1 Nef protein and peptides derived from the antigens of Plasmodium falciparum, such as the CS.T3 peptide (Sinigaglia et al., Nature, 1988, 336, 778-780) and the CSP, SSP2, LSA-1 and EXP-1 peptides (Doolan et al., J. Immunol., 2000, 165, 1123-1137).

The combination of the CD4+ T epitope with at least one of the epitopes as defined above advantageously makes it possible to trigger or to modulate an anti-EBV immune response.

A subject of the present invention is also a lipopeptide, characterized in that it comprises a peptide or a polyepitope fragment as defined above. Said lipopeptide is in particular obtained by addition of a lipid to an α-amino function or to a reactive function of the side chain of an amino acid of said peptide or polyepitope fragment; it can comprise one or more chains derived from C₄₋₂₀ fatty acids, optionally branched or unsaturated (palmitic acid, oleic acid, linoleic acid, linolenic acid, 2-aminohexadecanoic acid, pimelautide, trimexautide) or a derivative of a steroid. The preferred lipid part is in particular represented by an N^{α}-acetyllysine N^{ε} (palmitoyl) group, also called Ac-K(Pam).

A subject of the present invention is also a fusion protein, characterized in that it consists of a heterologous protein or protein fragment, fused with a peptide or a polyepitope fragment as defined above. The peptide or the polyepitope fragment can be fused with the NH₂ or COOH end of said protein or inserted into the sequence of said protein.

For the purpose of the present invention, the term “heterologous protein”, relative to the peptides derived respectively from EBNA1, LMP1 and LMP2, is intended to mean proteins other than EBNA1, LMP1 and LMP2.

Advantageously, said fusion protein consists of an EBV peptide as defined above, fused with one of the chains of an HLA II molecule chosen from the HLA-DR1, HLA-DR3, HLA-DR4, HLA-DR7, HLA-DR11, HLA-DR13, HLA-DR15, HLA-DRB3, HLA-DRB4, HLA-DRB5 and HLA-DP4 molecules, preferably the beta chain, or else with a fragment thereof, corresponding to a soluble HLA II molecule, in particular a fragment corresponding to the extracellular domain preceded by the homologous signal peptide or by a heterologous signal peptide. Said EBV peptide is advantageously inserted between the signal peptide and the NH₂ end of the extracellular domain of the β chain, as described for the HLA-DR molecule (Kolzin et al., PNAS, 2000, 97, 291-296).

Alternatively, said EBV peptide or said polyepitope fragment is fused with a protein that facilitates its purification or its detection, known to those skilled in the art, such as, in particular, glutathione-S-transferase (GST) and fluorescent proteins (GFP and derivatives). In this case, the sequence of the peptide or of the polyepitope fragment of interest is preferably separated from the rest of the protein by a cleavage site for a protease, for facilitating the purification of said peptide or of said polyepitope fragment.

A subject of the present invention is also a polynucleotide, characterized in that it encodes a peptide, a polyepitope fragment or a fusion protein as defined above.

In accordance with the invention, the sequence of said polynucleotide is that of the cDNA encoding said peptide or polyepitope fragment or said fusion protein. Said sequence can advantageously be modified in such a way that the codon use is optimal in the host in which it is expressed.

The subject of the invention also encompasses the recombinant polynucleotides comprising at least one polynucleotide in accordance with the invention, linked to at least one heterologous sequence.

For the purpose of the present invention, the term “heterologous sequence”, relative to a nucleic acid sequence encoding an EBV peptide, is intended to mean any nucleic acid sequence other than those which, naturally, are immediately adjacent to said nucleic acid sequence encoding said EBV peptide.

The subject of the present invention encompasses, in particular:

a) expression cassettes comprising at least one polynucleotide as defined above, under the control of appropriate regulatory sequences for transcription and, optionally, translation (promoter, activator, intron, initiation codon (ATG), stop codon, polyadenylation signal), and
b) recombinant vectors comprising a polynucleotide in accordance with the invention. Advantageously, these vectors are expression vectors comprising at least one expression cassette as defined above.

A subject of the present invention is also prokaryotic or eukaryotic host cells modified with at least one polynucleotide or one vector as defined above.

Numerous vectors into which it is possible to insert a nucleic acid molecule of interest in order to introduce it and to maintain it in a eukaryotic or prokaryotic host cell are known in themselves; the choice of an appropriate vector depends on the use envisioned for this vector (for example, replication of the sequence of interest, expression of this sequence, maintenance of this sequence in extrachromosomal form, or else integration into the host's chromosomal material) and also on the nature of the host cell. For example, it is possible to use, inter alia, viral vectors such as adenoviruses, retroviruses, lentiviruses, AAVs and baculoviruses, into which the sequence of interest has been inserted beforehand; it is also possible to associate said sequence (isolated or inserted into a plasmid vector) with a substance which allows it to cross the host cell membrane, such as a transporter, for instance a nanotransporter, or a preparation of liposomes or of cationic polymers, or else to introduce it into said host cell using physical methods such as electroporation or microinjection. In addition, these methods can advantageously be combined, for example by using electroporation associated with liposomes.

The polynucleotides, the recombinant vectors and the transformed cells as defined above are useful in particular for the production of the peptides, polyepitope fragments and fusion proteins according to the invention.

The polynucleotides according to the invention are obtained by conventional methods, known in themselves, according to standard protocols such as those described in Current Protocols in Molecular Biology (Frederick M. Ausubel, 2000, Wiley and Son Inc., Library of Congress, USA). For example, they can be obtained by amplification of a nucleic sequence by PCR or RT-PCR, by screening genomic DNA libraries by hybridization with a homologous probe, or else by total or partial chemical synthesis. The recombinant vectors are constructed and introduced into host cells by conventional recombinant DNA and genetic engineering methods, which are known in themselves.

The peptides and their derivatives (variants, modified peptides, lipopeptides, polyepitope fragments, fusion proteins) as defined above are prepared by the conventional techniques known to those skilled in the art, in particular by solid-phase or liquid-phase synthesis or by expression of a recombinant DNA in an appropriate cell system (eukaryotic or prokaryotic).

More specifically,
the peptides and their derivatives (variants, polyepitope fragments) can be solid-phase synthesized, according to the Fmoc technique, originally described by Merrifield et al. (J. Am. Chem. Soc. 1964, 85: 2149-) and purified by reverse-phase high performance liquid chromatography,
the lipopeptides can in particular be prepared according to the method described in international applications WO 99/40113 or WO 99/51630,
the peptides and derivatives such as the variants, the polyepitope fragments and fusion proteins can also be produced from the corresponding cDNAs, obtained by any means known to those skilled in the art; the cDNA is cloned into a eukaryotic or prokaryotic expression vector and the protein or the fragment produced in the cells modified with the recombinant vector is purified by any appropriate means, in particular by affinity chromatography.

A subject of the present invention is also an immuno-genic or vaccinal composition, characterized in that it comprises at least one EBV peptide, one polyepitope fragment, one lipopeptide or one vector as defined above, and a pharmaceutically acceptable carrier, and/or a carrier substance, and/or an adjuvant.

The immunogenic composition according to the invention is in a galenic form suitable for parenteral (subcutaneous, intramuscular, intravenous), enteral (oral, subligual), or local (rectal, vaginal) administration.

The pharmaceutically acceptable carriers, the carrier substances and the adjuvants are those conventionally used.

The adjuvants are advantageously chosen from the group consisting of: oily emulsions, mineral substances, bacterial extracts, saponin, alumina hydroxide, monophosphoryl lipid A, and squalene.

The carrier substances are advantageously selected from the group consisting of: unilamellar or multilamellar liposomes, ISCOMs, virosomes, viral pseudo-particles, saponin micelles, solid microspheres which are saccharide (poly(lactide-co-glycolide)) or gold-bearing in nature, and nanoparticles.

Preferably, said immunogenic composition comprises at least two of the six EBV peptides including a CD4+ T epitope as defined above, preferably three to five peptides, preferably the six peptides, in the form of a mixture of peptides or of lipopeptides, of a polyepitope fragment or of an expression vector encoding said peptides or said fragment.

Preferably, said composition also comprises at least one other peptide including an epitope selected from the group consisting of: a universal CD4+ T epitope or an EBV CD8+ T epitope, as defined above.

The subject of the present invention is also a peptide, a polyepitope fragment, a lipopeptide or a vector as defined above, as a vaccine for the prevention and/or treatment of an EBV infection or of an associated tumoral pathology.

A subject of the present invention is also the use of a peptide, of a polyepitope fragment, of a lipopeptide or of a vector as defined above, for the preparation of a vaccine for use in the prevention and/or treatment of an EBV infection or of an associated tumoral pathology.

The peptides according to the present invention and the derived products (polyepitope fragment, lipopeptide, recombinant vector) can be used in immunotherapy in the treatment of EBV-associated tumors, and in particular those exhibiting a type I or II latency. Said peptides or derived products are used either as a vaccine or in cell therapy, or alternatively through a combination of the two approaches.

The cell therapy comprises the preparation of EBV-specific CD4+ T lymphocytes by means of a conventional in vitro stimulation protocol comprising the isolation of peripheral blood mononuclear cells (PBMC) from a patient to be treated or from a volunteer donor of identical or partially identical HLA phenotype, and the culturing of the PBMCs in the presence of peptide(s), so as to induce the proliferation of EBV-specific CD4+ T lymphocytes. In a second step, the EBV-specific CD4+ T lymphocytes are reinjected into the patient.

A subject of the present invention is also a reagent for diagnosing and monitoring the evolution of an EBV infection or of an associated tumoral pathology, characterized in that it comprises at least one peptide as defined above, optionally labeled or complexed, in particular complexed with labeled HLA II molecules, in the form of multimeric complexes such as tetramers.

A subject of the present invention is also the use of a peptide as defined above, for the preparation of a reagent for diagnosing and monitoring the evolution of an EBV infection or of an associated tumoral pathology.

For the purpose of the present invention, the expression “diagnosing or monitoring the evolution of an EBV infection or of an associated tumoral pathology” is intended to mean the evaluation of the EBV-specific CD4+ T immune response over the course of an EBV infection, of an EBV-associated pathology, or else of an anti-EBV immunotherapy or immunization protocol. The detection is carried out using a biological sample containing CD4+ T cells, in particular a sample of mononuclear cells isolated from a peripheral blood sample (PBMCs).

A subject of the present invention is also a method of diagnosing and monitoring the evolution of an EBV infection or of an associated tumoral pathology, characterized in that it comprises:
bringing a biological sample from said individual into contact with a diagnostic reagent as defined above, and
detecting CD4+ T lymphocytes specific for one of the EBNA1, LMP1 or LMP2 latency antigens of EBV, by any appropriate means.

A subject of the present invention is also a kit for detecting and monitoring the evolution of an EBV infection or of an associated tumoral pathology, characterized in that it comprises at least one reagent as defined above, combined with a means of detecting CD4+ T lymphocytes specific for one of the EBNA1, LMP1 and LMP2 latency antigens of EBV.

The detection of the CD4+ T lymphocytes specific for a latency antigen is carried out by any means, known in themselves. For example, it is possible to use direct means, such as lymphocyte proliferation assays or flow cytometry in the presence of multimeric complexes as defined above, or else indirect means, for instance the assaying of cytokines such as IL-2, IL-4, IL-5, IL-10 and IFN-γ, in particular by immunoenzymatic techniques (ELISA, RIA, ELISPOT) or by flow cytometry (assaying of intracellular cytokines).

More specifically:

A suspension of cells (PBMCs, PBMCs depleted of CD8+ cells, T lymphocytes pre-enriched by means of an in vitro culture step with the peptides as defined above or cloned T lymphocytes) is cultured for 3 to 5 days in the presence of said peptides and, as required, of appropriate presenting cells, such as dendritic cells, autologous or heterologous PBMCs, lymphoblastoid cells such as those obtained after infection with the EBV virus, or genetically modified cells. The presence of CD4+ T cells specific for an EBV latency antigen in the initial suspension is detected by means of the EBV peptides, according to one of the following methods:

Proliferation Assay:

The proliferation of the CD4+ T cells specific for an EBV latency antigen is measured by incorporation of tritiated thymidine into the DNA of the cells.

ELISPOT Assay:

The ELISPOT assay makes it possible to reveal the presence of T cells secreting cytokines (IL-2, IL-4, IL-5, IL-10 and IFN-γ), specific for a peptide as defined above. The principle of this assay is described in Czerkinsky et al., J. Immunol. Methods, 1983, 65, 109-121 and Schmittel et al., J. Immunol. Methods, 1997, 210, 167-174, and its implementation is illustrated in international application WO 99/51630 or Gahéry-Ségard et al., J. Virol., 2000, 74, 1694-1703.

Detection of Cytokines:

The presence of T cells specific for an EBV latency antigen, secreting cytokines such as IL-2, IL-4, IL-5, IL-10 and IFN-γ is detected either by assaying the cytokines present in the culture supernatant, by means of an immunoenzymatic assay, in particular using a commercial kit, or by detecting the intracellular cytokines by flow cytometry. The principle of detection of the intracellular cytokines is described in Goulder et al., J. Exp. Med., 2000, 192, 1819-1832 and Maecker et al., J. Immunol. Methods, 2001, 225, 27-40 and its implementation is illustrated in Draenert et al., J. Immunol. Methods, 2003, 275, 19-29.

Multimeric Complexes
a biological sample, preferably peripheral blood mononuclear cells (PBMCs), is brought into contact with labeled multimeric complexes, in particular labeled with a fluorochrome, formed by binding between soluble HLA II molecules and EBV peptides as defined above, and
the cells labeled with said multimeric complexes are analyzed, in particular by flow cytometry.

Advantageously, prior to the biological sample being brought into contact with said complexes, it is enriched in CD4+ T cells by bringing it into contact with CD4+ T antibodies or by indirect sorting, in order to prevent nonspecific activation.

The HLA II/peptide multimeric complexes can be prepared from natural molecules extracted from cells expressing HLA II or from recombinant molecules produced in appropriate host cells as specified, for example, in Novak et al. (J. Clin. Investig., 1999, 104, R63-R67) or in Kuroda et al. (J. Virol., 2000, 74, 18, 8751-8756). These HLA II molecules can in particular be truncated (deletion of the transmembrane domain) and their sequence can be modified in order to make them soluble or else to facilitate the pairing of the alpha and beta chains (Novak et al., mentioned above).

The loading of HLA II molecules with the peptide can be carried out by bringing a preparation of HLA II molecules as above into contact with the peptide. For example, biotinylated, soluble HLA II molecules are incubated, for 72 hours at 37° C., with a 10-fold excess of EBV peptides as defined above, in a 10 mM phosphate-citrate buffer containing 0.15M NaCl, at a pH of between 4.5 and 7.

Alternatively, the sequence of the peptide can be introduced into one of the chains of the HLA II molecule in the form of a fusion protein which allows the preparation of HLA II/peptide multimeric complexes from appropriate host cells expressing said fusion protein. Said complexes can then be labeled, in particular with biotin.

The multimeric complexes of tetramer type are in particular obtained by adding, to the loaded HLA II molecules, streptavidin labeled with a fluorochrome in an amount four times less (mole for mole) with respect to the HLA II molecules, the whole mixture then being incubated for a sufficient period of time, for example overnight at ambient temperature.

The multimeric complexes can also be formed either by incubation of HLA II/peptide monomers with magnetic beads coupled to streptavidin, as described for HLA I molecules (Bodinier et al., Nature 2000, 6, 707-710), or by insertion of HLA II/peptide monomers into lipid vesicles as described for murine class II MHC molecules (Prakken, Nature Medicine, 2000, 6, 1406-1410).

To use these HLA II/peptide multimeric complexes, in particular of tetramer type, a suspension of cells (PMBCs, PBMCs depleted of CD8+ cells, T lymphocytes pre-enriched by means of an in vitro culture step with EBV peptides as defined above, or cloned T lymphocytes) into contact with HLA II/peptide multimeric complexes at an appropriate concentration (for example of the order of 10 to 20 μg/ml), for a period of time sufficient to allow binding between the complexes and the EBV-specific CD4+ T cells (for example, of the order of 1 to 3 hours). After washing, the suspension is analyzed by flow cytometry: the labeling of the cells is visualized by means of the multimeric complexes which are fluorescent.

The flow cytometry makes it possible to separate the cells labeled with the HLA II/peptide multimeric complexes from the unlabeled cells and to thus perform cell sorting.

A subject of the present invention is thus also a method of sorting CD4+ T cells specific for an EBV latency antigen, characterized in that it comprises at least the following steps:
bringing a cell sample into contact with labeled HLA II/peptide multimeric complexes, in particular labeled with a fluorochrome, said complexes being formed by the binding of soluble HLA II molecules with at least one EBV peptide as defined above, and
sorting the cells bound to said HLA II/peptide complexes, in particular by flow cytometry.

In addition to the above arrangements, the invention also comprises other arrangements, which will emerge from the description which follows, which refers to examples of implementation of the subject of the present invention, with reference to the attached drawings in which:

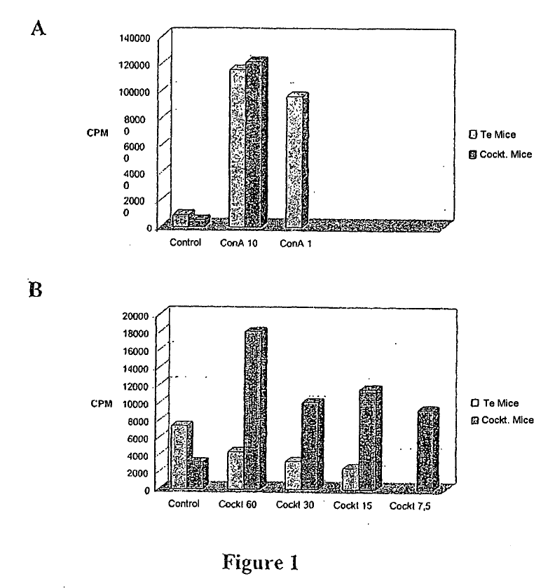

FIG. 1 illustrates the immunogenicity of the mixture of the six peptides derived from the EBV type I and II latency antigens binding with an affinity<1000 nM to the 12 HLA II molecules predominant in the Caucasian population, in the HLA-DR1 transgenic murine model. A group of five HLA-transgenic Aβ°/DR1 mice (Cockt mice) was immunized with a first subcutaneous injection of the peptide mixture (120 μg in total) emulsified in Montanide® Isa 720, followed by 2 half-dose boosters, 2 weeks apart. The control group (Te mice) received only Montanide® Isa 720, according to the same protocol. The proliferative response of the lymphocytes extracted from the lymph nodes, 7 days after the final injection, was measured by incorporation of (³H)thymidine, under various conditions. A: in the absence of antigen (control) or in the presence of a concanavalin A (ConA) range (48-hour cultures). B: in the absence of antigen (control) or in the presence of a range of concentrations of the peptide mixture (60, 30, 15 or 7.5 μg in total) (5-day cultures). The results are expressed as number of counts per minute (cpm);

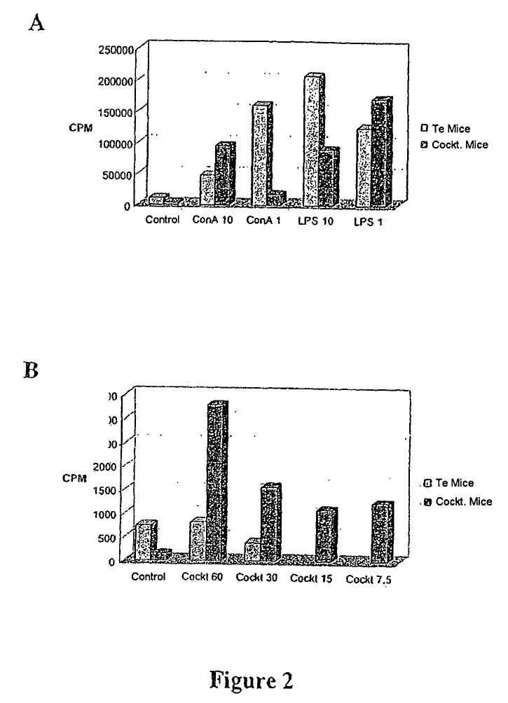

FIG. 2 illustrates the immunogenicity of the mixture of the six peptides derived from the EBV type I and II latency antigens binding with an affinity<1000 nM to the 12 HLA II molecules predominant in the Caucasian population, in the HLA-DR1 transgenic murine model. A group of five HLA-transgenic Aβ°/DR1 mice (Cockt mice) was immunized with a first subcutaneous injection of the peptide mixture (120 μg in total) emulsified in Montanide® Isa 720, followed by 2 half-dose boosters, 2 weeks apart. The control group (Te mice) received only Montanide® Isa 720, according to the same protocol. The proliferative response of the lymphocytes extracted from the spleens, 7 days after the final injection, was measured by incorporation of (³H)thymidine, under various conditions. A: in the absence of antigen (control) or in the presence of a concanavalin A (ConA) or LPS range (48-hour cultures). B: in the absence of antigen (control) or in the presence of a range of concentrations of the peptide mixture (60, 30, 15 or 7.5 μg in total) (5-day cultures). The results are expressed as number of counts per minute (cpm);

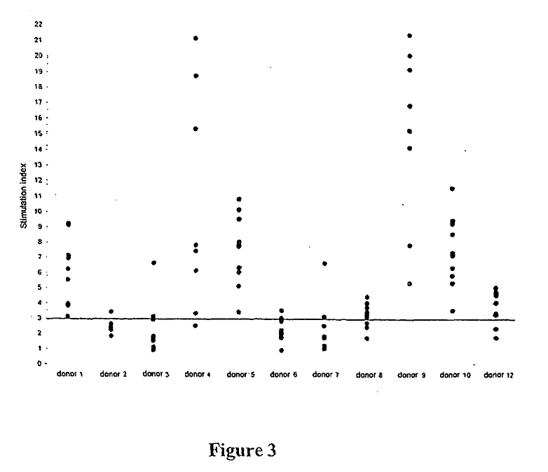

FIG. 3 illustrates the proliferative response of the T lymphocytes derived from normal donors of varied HLA II haplotypes, who are seropositive for EBV, after in vitro stimulation with the peptide mixture. The blood mononuclear cells derived from normal individuals seropositive for EBV (96-well plates, 5×10⁵ cells per well) were cultured for 4 to 6 days in the presence of the mixture containing the peptides G17F, N26E, V21V, K26L, P24G and I16L (SEQ ID NOS: 21, 4, 20, 5, 6 and 11) at the concentration of 10 μg/ml for each of them. The proliferative response of the T lymphocytes was measured by incorporation of (³H)thymidine. The stimulation index corresponding to the ratio of the number of counts per minute of the T lymphocytes in the presence of the peptide mixture, to the average number of counts per minute of the T lymphocytes in the absence of peptide (background noise), is presented for each well for the various donors. A well is considered to be positive when the number of counts per minute is greater than 1000 and the stimulation index greater than 3.

### EXAMPLE 1

### Identification of CD4+ T Epitopes Recognized by the HLA II Molecules Most Abundant in the Caucasian Population

### 1) Materials and Methods

### 1.1) Peptide Synthesis

The TEPITOPE software (Sturniolo et al., Nature Biotechnology, 1999, 17, 555-561) was used to select potential class II HLA molecule binding motifs in the sequence of the EBV type II latency antigens (EBNA1, LMP1 and LMP2). The corresponding peptides were synthesized according to the Fmoc strategy in parallel synthesis on a solid phase (Neosystem), purified by high performance liquid chromatography (HPLC; C18 column, Symmetry), and then controlled by mass spectrometry (ES-MS).

### 1.2) HLA II/Peptide Binding Assays

The assays for binding to HLA II molecules are competition binding assays with immunoenzymatic revelation, the principle of which is described in U.S. Pat. No. 6,649,169 for HLA-DR molecules and in PCT international application WO 03/040299 for HLA-DP4 molecules, and also in the articles under the names of Texier et al., J. Immunol., 2000, 164, 3177 and Eur. J. Immunol., 2001, 31, 1837 and Castelli et al., J. Immunol., 2002, 169, 6928-6934.

Examples of these assays are given in PCT international applications WO 02/090382, WO 03/040299 and WO 2004/014936.

### a) Choice of HLA II Alleles

12 HLA II molecules (10 HLA-DR molecules and 2 HLA-DP molecules) most abundant in the French population, and the allelic frequencies of which are characteristic of the Caucasian population, were selected:

HLA-DR Molecules in which the β Chain is Encoded by the DR1 Gene

They are the HLA-DR1, -DR3, -DR4, -DR7, -DR11, -DR13 and -DR15 molecules in which the β chain is encoded by the alleles of the DRB1 locus, the frequency of which exceeds 5% in the French population: DRB1*0101 (9.3%), DRB1*0301 (10.9%), DRB1*0401 (5.6%), DRB1*0701 (14%), DRB1*1101 (9.2%), DRB1*1301 (6%) and DRB1*1501 (8%), which by themselves represent 64% of the population. These same alleles are the HLA-DR alleles most abundant in the other Caucasian populations. Their frequency varies between 53% (in Spain) and 82% (in Denmark). For the United States and Canada, they represent 58% and 55%, respectively, of the DR alleles in the population.

HLA-DR Molecules in which the βChain is not Encoded by the DR1 Gene

They are the HLA-DRB3, -DRB4 and -DRB5 molecules in which the β chain is encoded by the alleles most common in the French population: HLA-DRB3*0101 (9.2%), HLA-DRB4*0101 (28.4%) and HLA-DRB5*0101 (7.9%), These molecules by themselves cover 45% of the allelic frequency.

HLA-DP Molecules

They are the HLA-DP4 molecules which group together the molecules encoded by the DPB1*0401 and DPB1*0402 alleles. These DP4 molecules are the HLA II molecules most abundant in Europe and the United States. Their allelic frequency is in fact 40% and 11%, respectively, which means that one or other of them is found in approximately 76% of individuals. The peptides present in a protein sequence and which bind all these molecules therefore include the CD4+ T epitopes of the majority of the population.

### b) Purification of HLA II Molecules

The HLA II molecules are purified by immunoaffinity from various homozygous lines of human B lymphocytes transformed with the Epstein-Barr virus (EBV), namely: HOM2 (DRB1*0101, DPB1*0401), SCHU (DRB1*1501, DRB5*0101, DPB1*0402), STEILIN (DRB1*0301, DRB3*0101), PITOUT (DRB1*0701, DBR4*0101, DPB1*0401), SWEIG (DRB1*1101), BOLETH (DRB1*0401, DRB4*0103), HHKB (DRB1*1301, DRB3*0101, DPB1*0401). The HLA-DR molecules are purified by affinity chromatography using the L243 monomorphic monoclonal antibody (Smith et al., P.N.A.S., 1982, 79, 608-612) coupled to protein A-sepharose CL 4B gel (Pharmacia), as described in Gorga et al., J. Biol. Chem., 1987, 262, 16087-16094. The HLA-DP4 molecules are purified according to the same protocol, using the B7/21 monomorphic antibody (Watson et al., Nature, 1983, 304, 358-361). Briefly, the cells (5×10⁸ cells/ml) are lysed on ice, in a buffer of 150 nM NaCl, 10 mM Tris HCl, pH=8.3, containing 1% Nonidet P40, 10 mg/l aprotinin, 5 mM EDTA and 10 μM PMSF. After centrifugation at 100 000 g for 1 h, the supernatant is loaded onto the sepharose 4B and protein A-sepharose 4B columns and then onto the specific affinity column. The HLA II molecules are eluted with a buffer of 1.1 mM n-dodecyl-β-D-maltoside (DM), 500 mM NaCl and 500 mM Na₂CO₃, pH=11.5. The fractions are immediately neutralized to pH=7 with a 2M Tris HCl buffer, pH=6.8, and then extensively dialyzed against a buffer of 1 mM DM, 150 mM NaCl, 10 mM, pH=7.

### c) Synthesis of Tracer Peptides

The tracer peptides used in the binding assays are the following: HA 306-318 (PKYVKQNTLKLAT, SEQ ID NO: 23), A3 152-166 (EAEQLRAYLDGTGVE, SEQ ID NO: 24), MT 2-16 (AKTIAYDEEARRGLE, SEQ ID NO: 25), YKL(AAYAAAKAAALAA, SEQ ID NO: 26), B1 21-36 (TERVRLVTRHIYNREE, SEQ ID NO: 27), LOL 191-210 (ESWGAVWRIDTPDKLTGPFT, SEQ ID NO: 28), E2/E168 (AGDLLAIETDKATI, SEQ ID NO: 29) and Oxy 271-287 (EKKYFAATQFEPLAARL, SEQ ID NO: 30). The peptides are synthesized according to the Fmoc strategy in parallel synthesis on a solid phase, biotinylated at the terminal NH₂ residue using biotinyl-6-aminocaproic acid (Fluka Chimie), according to the protocol described in Texier et al., J. Immunol., 2000, 164, 3177-3184), and then cleaved from the resin with trifluoroacetic acid (95%) and purified by reverse-phase high performance liquid chromatography on a C₁₈ column (Vydac™).

### d) HLA II/Peptide Binding Assays

The HLA II molecules are diluted in 10 mM phosphate buffer containing 150 mM NaCl, 1 mM dodecyl maltoside (DM), 10 mM citrate and 0.003% thimerosal, in the presence of an appropriate biotinylated peptide (tracer peptide), at a given pH, and of serial dilutions of competitive peptide (peptide to be tested). Namely: the HA 306-318 peptide is used at 1 nM and 30 nM, at pH=6, respectively for the DRB1*0101 and DRB1*0401 alleles, and at 20 nM, at pH=5, for the DRB1*1101 allele. YKL is used at 20 nM, at pH=5, for the DRB1*0701 allele. The incubation is carried out at pH=4.5 for the DRB1*1501, DRB1*1301 and DRB1*0301 alleles, in the presence of, respectively, A3 152-166 (10 mM), B1 21-36 (200 nM) and MT 2-16 (200 nM). The oxybiotinylated peptide is used at pH=5 (10 nM) for the DPB1*0401 and DPB1*0402 alleles. The reaction mixture (100 μl per well) is incubated in polypropylene 96-well plates (NUNC), at 37° C. for 24 h, with the exception of the DRB1*1301 and DRB1*0301 alleles, which are incubated for 72 h. At the end of the incubation, the samples are neutralized with 50 μl of 450 mM Tris HCl buffer, pH 7.5, containing 0.003% thimerosal, 0.3% BSA and 1 mM DM. The samples are then transferred onto Maxisorp™ ELISA plates (96-well, NUNC) pre-coated with antibodies L243 or B7/21 L243 (10 μg/ml) and saturated with 100 mM Tris HCl buffer, pH=7.5, containing 0.3% BSA and 0.003% of thimerosal. The incubation of the samples on these plates is carried out for 2 hours at ambient temperature. Washes with 0.1M Tris HCl buffer, pH 7.5, containing 0.05% Tween-20 are then carried out between each step. The biotinylated peptide bound to the HLA II molecules is detected by the addition of 100 μL/well of the streptavidin-alkaline phosphatase conjugate (45 minutes, Amersham) diluted to 1/2000 in 10 mM Tris buffer, pH 7, containing 0.15M NaCl, 0.05% Tween 20, 0.2% BSA and 0.003% thimerosal, and then of 200 μL/well of the substrate 4-methylumbelliferyl-phosphate (MUP, SIGMA) at the concentration of 100 μM, in 0.05M NaHCO₃ buffer, pH 9.8, containing 1 mM MgCl₂. The emission of fluorescence by the product of the enzymatic reaction is measured at 450 nm after excitation at 365 nm, using a fluorimeter for 96-well plates (Dynex). The maximum binding is determined by incubating the biotinylated tracer peptide with the HLA II molecule in the absence of competitor peptide. The binding specificity is controlled by the addition of an excess of nonbiotinylated peptide. The background noise obtained does not significantly differ from that obtained by incubating the biotinylated peptide without the HLA II molecules. The results are expressed in the form of the concentration of competitor peptide which inhibits 50% of the maximum binding of the biotinylated tracer peptide (IC₅₀). The means and the standard deviations are calculated from at least three independent experiments. The validity of each experiment is determined using the tracer peptides: their IC₅₀ values vary by a factor of less than 3. A peptide whose IC₅₀ value is less than 1000 mM is considered to have a good affinity for the corresponding HLA II molecule.

### 2) Results

The analysis of the sequences of the EBNA1, LMP1 and LMP2 antigens made it possible to identify 24 peptide sequences of 15 to 31 amino acids, liable to contain motifs for several HLA-DR molecules. Among these 24 peptides, 22 could be synthesized by f-moc chemistry with a purity of the order of 90% by HPLC. Nine peptides are derived from EBNA1, 7 from LMP1 and 6 from LPM2. The sequences of the peptides derived from the sequences having the accession numbers NP_{—}039875, CAA26023 and AAA45886, respectively for EBNA1, LMP1 and LMP2, are given in the sequence listing attached in the annex and table II.

TABLE II

Peptide sequences

| | | | Identifier |
|---|---|---|---|
| Peptide | Position | Sequence | number |
| EBNA1 | | | |
| R19T | 67-85 | RDGVRRPQKRPSCIGCKGT | SEQ ID NO: 1 |
| R26D | 403-428 | RPFFHPVGEADYFEYHQEGGPDGEPD | SEQ ID NO: 2 |
| D22P | 455-476 | DGGRRKKGGWFGKHRGQGGSNP | SEQ ID NO: 3 |
| N26E | 475-500 | NPKFENIAEGLRALLARSHVERTTDE | SEQ ID NO: 4 |
| K26L | 514-539 | KTSLYNLRRGTALAIPQCRLTPLSRL | SEQ ID NO: 5 |
| P24G | 529-552 | PQCRLTPLSRLPFGMAPGPGPQPG | SEQ ID NO: 6 |
| M23T | 563-585 | MVFLQTHIFAEVLDAIKDLVMT | SEQ ID NO: 7 |
| F24R | 571-594 | FAEVLKDAIKDLVMTKPAPTCNIR | SEQ ID NO: 8 |
| L19G | 606-624 | LPPWFPPMVEGAAAEGDDG | SEQ ID NO: 9 |
| LMP1 | | | |
| E15P | 2 to 16 | EHDLERGPPGPRRPP | SEQ ID NO: 10 |
| I16L | 68-83 | IIFIFRRDLLCPLGAL | SEQ ID NO: 11 |
| A16L | 96-111 | ALWNLHGQALFLGIVL | SEQ ID NO: 12 |
| A17H | 179-195 | AILIWMYYHGPRHSDEH | SEQ ID NO: 13 |
| H15D | 303-317 | HDPLPHSPSDSAGND | SEQ ID NO: 14 |
| 527L | 211 to 237 | SGHESDSNSNEGRHHLLVSGAGDGPPL | SEQ ID NO: 15 |
| D31D | 356 to 386 | DPHLPTLLLGTSGSGGDDDDPHGPVQLSYYD | SEQ ID NO: 16 |
| LMP2 | | | |
| M20D | 1 to 20 | MGSLEMVPGAGPPSPGGDP | SEQ ID NO: 17 |
| E25Y | 61 to 85 | EDPYWGNGDRHSDYQPLGTQDQSLY | SEQ ID NO: 18 |
| A1SV | 148-163 | ASSYAAAQRKLLTPV | SEQ ID NO: 19 |
| V21V | 224-244 | VLVMLVLLILAYRRRWRRLTV | SEQ ID NO: 20 |
| G17F | 372-388 | GGSILQTNFKSLSSTEF | SEQ ID NO: 21 |
| L18L | 410-427 | LTEWGSGNRTYGPVFMCL | SEQ ID NO: 22 |

The binding activity of the peptides with respect to the 12 HLA II molecules predominant in the Caucasian population (HLA-DR1, -DR3, -DR4, -DR7, -DR1, -DR13, -DR15, -DRB3, -DRB4, -DRB5, DP401 and -DP402; table III) shows that 6 peptides bind with good affinity to at least 7 different HLA II molecules: G17F (LMP2), N26E (EBNA1), V21V (LMP2), K26L (EBNA1), P24G (EBNA1), I16L (LMP1).

TABLE III
Binding activity of the peptides with respect to the HLA II
molecules predominant in the Caucasian population
IC₅₀ *(nM)

| Peptide | DR1 | DR3 | DR4 | DR7 | DR11 | DR13 | DR15 | DRB3 |
|---|---|---|---|---|---|---|---|---|
| L18L | Insoluble | Insoluble | >100000 | 1061 | >100000 | Insoluble | Insoluble | Insoluble |
| G17F | 15 | 851 | 335 | 40 | 29 | Insoluble | 28 | >100000 |
| N26E | 21 | 8485 | 235 | 184 | 447 | 1649 | 229 | >100000 |
| A17H | 7746 | 7348 | >100000 | >100000 | >100000 | Insoluble | 3 | >100000 |
| A16L | 79 | Insoluble | >100000 | 122 | 10954 | Insoluble | 48 | >100000 |
| M20P | 721 | >100000 | 7483 | >100000 | 4243 | >100000 | 4690 | >100000 |
| L19G | 6221 | >100000 | >100000 | 18166 | 1095 | >100000 | 69354 | >100000 |
| S27L | >100000 | >100000 | >100000 | 18000 | 19339 | >100000 | 45000 | >100000 |
| D31D | 17321 | >100000 | >100000 | 27495 | 25000 | >100000 | 251 | >100000 |
| A15V | 1196 | >100000 | 35496 | 1732 | 4 | >100000 | 26458 | >100000 |
| E25Y | 949 | >100000 | 5099 | 9798 | 2258 | >100000 | 1285 | >100000 |
| V21V | 240 | 77 | 390 | 71 | 130 | 161 | 693 | 417 |
| RT19 | 698 | >100000 | 9899 | 38536 | 11292 | >100000 | 7266 | >100000 |
| K26L | 1.1 | 60000 | 346 | 1.7 | 1 | 2049 | 24 | >100000 |
| R26D | 3742 | >100000 | 20494 | 1129 | >100000 | >100000 | 22361 | 3969 |
| P24G | 47 | 32787 | 2627 | 116 | 2 | 775 | 2621 | 31464 |
| F24R | 65 | 1936 | 1500 | 56 | 1554 | >100000 | 3464 | 16125 |
| D22P | 62450 | >100000 | >100000 | 86603 | 229 | >100000 | >100000 | >100000 |
| I16L | 190 | 110 | 7348 | 175 | 27 | >100000 | 9 | 224 |
| E15P | >100000 | >100000 | >100000 | >100000 | >100000 | >100000 | >100000 | >100000 |
| H15D | >100000 | >100000 | >100000 | >100000 | >100000 | >100000 | >100000 | >100000 |

| | | | | | Number | |
|---|---|---|---|---|---|---|
| | | | | | of alleles | |
| Peptide | DRB4 | DRB5 | DP401 | DP402 | bound | Classification |
| L18L | 2100 | Insoluble | Insoluble | Insoluble | 0 | 17 |
| G17F | Insoluble | 170 | 16 | 25 | 9 | 2 |
| N26E | 825 | 17 | 4094 | 4402 | 7 | 4 |
| A17H | 150 | 42 | >100000 | >100000 | 3 | 8 |
| A16L | >100000 | 2683 | 1182 | 725 | 4 | 7 |
| M20P | 6735 | 1039 | 11745 | 10595 | 1 | 12 |
| L19G | >100000 | 37417 | >100000 | 30984 | 0 | 17 |
| S27L | >100000 | 13477 | >100000 | >100000 | 0 | 17 |
| D31D | >100000 | >100000 | >100000 | 28284 | 1 | 12 |
| A15V | >100000 | 463 | >100000 | >100000 | 2 | 9 |
| E25Y | >100000 | >100000 | >100000 | >100000 | 1 | 12 |
| V21V | 167 | 169 | 150 | 155 | 12 | 1 |
| RT19 | >100000 | 771 | >100000 | >100000 | 2 | 9 |
| K26L | 200 | 128 | 194 | 339 | 9 | 2 |
| R26D | 79373 | 5477 | >100000 | >100000 | 0 | 17 |
| P24G | 5532 | 419 | 120 | 447 | 7 | 4 |
| F24R | 63246 | 5399 | 17544 | 2739 | 2 | 9 |
| D22P | >100000 | >100000 | >100000 | >100000 | 1 | 12 |
| I16L | 529 | >100000 | >100000 | 1111 | 7 | 4 |
| E15P | >100000 | 5254 | >100000 | >100000 | 0 | 17 |
| H15D | >100000 | >100000 | >100000 | >100000 | 0 | 17 |

*a peptide which has an IC₅₀ value of less than 1000 nM is considered to have a good affinity for the corresponding HLA II molecule

Given the allelic frequency of each of the 12 HLA II alleles tested, a mixture containing these 6 peptides could be recognized by more than 90% of the Caucasian population.

### EXAMPLE 2

### Immunogenicity of the Peptides In Vivo in Mice Transgenic for an HLA II Molecule

The ability of the peptides with a good affinity for the HLA II molecules, as defined in example 1, to induce an immune response was evaluated by means of an in vitro proliferation assay in mice transgenic for the HLA-DR1 molecules (Aβ°/DR1 transgenic mice; Pancré et al., Clinical and Experimental Immunology, 2002; 129: 429-437).

### 1) Materials and Methods

A first group of 6-week-old transgenic mice was immunized with a first subcutaneous injection of the peptide mixture determined in example 1 (20 μg of each peptide, i.e. 120 μg in total) emulsified in Montamide® Isa 720 (Seppic), followed by two boosters 2 weeks apart, with a half-dose of the peptide mixture (10 μg of each peptide, 60 μg in total) emulsified in Montanide® Isa 720. The control group received only injections of Montanide® Isa 720, according to the same protocol. Seven days after the final injection, the animals were sacrificed and the spleen and lymph nodes were removed. The spleen cells or lymph node cells (5×10⁵/well of a 96-well microtitration plate (Falcon®, in triplicate) were placed in culture, either in the absence of antigen (negative control), or in the presence of a range of LPS (Sigma®, 10 mg and 1 mg) or of concanavalin A (Sigma®, 10 mg and 1 mg) (positive controls, 48 h cultures), or in the presence of a range of concentrations (60 μg, 30 μg, 15 μg, 7.5 μg in total) of the peptide mixture (5-day cultures). After culturing, tritiated thymidine ((³H)thymidine) (1 mCi/well, 49.0 Ci/mmol, Amersham) was added and the cells were recovered 6 hours later by suction through a glass fiber filter (Wallac). The incorporated thymidine was measured using a β-scintillation counter (1450 Trilux, Wallac). The results were expressed as proliferation index (number of counts per minute (CPM) in the presence of antigen/number of CPM in the absence of antigen).

### 2) Results

The results presented in FIGS. 1 and 2 show that the peptides selected by means of the HLA II molecule-binding assay are capable of inducing a strong EBV-specific CD4+ response in immunized individuals. After restimulation in vitro with the peptide mixture, there is a proliferative response of the lymphocytes of the spleen and of the lymph nodes extracted from the immunized animals. On the other hand, this proliferation is not observed in the nonimmunized control animals (FIGS. 1 and 2). These results therefore indicate that the peptides selected can be used in immunogenic compositions for the prevention and treatment of pathologies associated with EBV infections.

### EXAMPLE 3

### Immunogenicity of the Peptides In Vitro in Normal Individuals Seropositive for EBV

### 1) Materials and Methods

### a) Donors and HLA Typing

Blood was taken from 12 normal volunteer donors seropositive for EBV and with a varied HLA II haplotype.

TABLE IV
HLA-DRB1 haplotypes of the normal volunteer
donors seropositive for EBV

| Donor | HLA-DRB1 haplotype |
|---|---|
| 1 | DRB1*1101/DRB1*1301 |
| 2 | DRB1*0401/DRB1*1201 |
| 3 | DRB1*1501/DRB1*0301 |
| 4 | DRB1*0701/DRB1*1101 |
| 5 | DRB1*0101/DRB1*1101 |
| 6 | DRB1*1501/DRB1*0401 |
| 7 | DRB1*0301/DRB1*1301 |
| 8 | DRB1*1101/DRB1*1201 |
| 9 | DRB1*1401/DRB1*0701 |
| 10 | DRB1*1101/DRB1*1201 |
| 11 | DRB1*0401/DRB1*1101 |
| 12 | DRB1*0401/DRB1*0801 |

### b) Proliferative Response of T Lymphocytes to the Peptide Mixture or to the Peptides Tested Individually

Blood mononuclear cells (PBMCs) from the donors were separated by the conventional Ficoll gradient method. The PBMCs (5×10⁵ cells/well of a 96-well plate) were cultured for 4 to 6 days in a serum-free medium (AIM V, Gibco™), in the presence or absence of the peptide mixture (60 μg/ml in total, i.e. 10 μg/mL of each peptide). The proliferative response was measured by incorporation of (³H)thymidine (1 mCi/well, 49.0 Ci/mmol, Amersham). The results are expressed as stimulation index (counts per minute of the stimulated T lymphocytes/mean of the counts per minute of the nonstimulated T lymphocytes). The wells corresponding to the stimulated T lymphocytes were analyzed individually and considered to be positive when the number of counts per minute was greater than 1000 and the stimulation index greater than 3 (number of counts per minute in the presence of the peptide mixture 3 times greater than that observed in the absence of peptides (background noise)). The peptides were also tested individually in 6 donors (donors 1, 3, 5, 6, 9 and 10). To do this, a range of concentrations (2.5, 5, and 20 μg) was tested in triplicate for each peptide. The response was considered to be positive when the mean of the stimulation index of the triplicates was greater than 2 with one well having a stimulation index greater than 3.

### c) Cytokine Detection

These PBMCs from the donors (1×10⁶ cells/well, 24-well plates) were cultured in AIM V medium (Gibco™) or RPMI 1640 medium (Gibco™), supplemented with 10% of group AB human serum, in the presence or absence of the peptide mixture (60 μg/mL), for 2 or 5 days. The IL-2, the IL-4, the IL-10 and the IFN-γ present in the culture supernatants were assayed by sandwich ELISA, according to a standard protocol, using antibodies directed against each of these cytokines (BD PharMingen). The absorbance at 492 nm was measured using a spectrophotometer (Multiskan RC, Labsystems). The results presented correspond to the mean obtained on two wells.

### 2) Results

### a) EBV-Specific CD4+ T Response Induced by the Peptide Mixture

In order to determine whether the peptide mixture was recognized by the human CD4+ T lymphocytes, blood mononuclear cells derived from normal volunteer donors seropositive for EBV, with a varied HLA haplotype (table III), were stimulated in vitro with the peptide mixture. Firstly, the proliferative response of the T lymphocytes, which makes it possible to establish the existence of a memory T lymphocyte response, was evaluated by measuring the incorporation of (³H)thymidine after 4 to 6 days of culture in the presence of the peptide mixture. The proliferative response could be evaluated in 11 donors (FIG. 3). All the donors show a proliferative response, with varying levels of proliferation from donor to the other. These results indicate the presence of memory T lymphocytes which can recognize the peptide mixture, in all the donors tested. The results also indicate that the mixture contains peptides naturally presented during EBV infection.

The response to each of the peptides tested separately was evaluated (table V). The results confirm the advantage of each of the 6 peptides in the mixture selected.

TABLE V
Analysis of the EBV-specific CD4+ T response
induced by each of the peptides tested separately

| | Peptides | | | | | | |
|---|---|---|---|---|---|---|---|
| | EBNA1 | | | LMP1 | LMP2 | | |
| Donors | N26E | K26L | P24G | I16L | G17F | V21V | |
| 1 | + | + | − | − | − | − | 2/6 |
| 3 | + | + | − | + | − | + | 4/6 |
| 5 | + | + | + | + | − | − | 4/6 |
| 6 | + | + | + | + | + | + | 6/6 |
| 9 | + | + | + | + | + | + | 6/6 |
| 10 | − | + | + | + | + | + | 5/6 |
| | 5/6 | 6/6 | 4/6 | 5/6 | 3/6 | 4/6 | |

The secretion of cytokines into the culture medium after stimulation of the PBMCs with the peptide mixture was also tested by ELISA (table VI).

TABLE VI
Analysis of cytokine secretion after stimulation with the peptide mixture

| | Cytokines (pg/ml) | | | | | | | |
|---|---|---|---|---|---|---|---|---|
| | IL-2 | | IL-4 | | IL-10 | | IFN-γ | |
| | AIM* | SAB** | AIM | SAB | AIM | SAB | AIM | SAB |
| Donor 1 | — | — | — | — | — | 80 | — | — |
| Donor 2 | — | — | — | — | — | 290 | — | 211 |
| Donor 3 | — | — | — | — | — | 645 | — | — |
| Donor 4 | — | — | — | — | — | — | — | — |
| Donor 5 | — | — | — | — | — | — | — | 220 |
| Donor 6 | — | — | — | — | — | — | 409 | 583 |
| Donor 7 | — | — | — | — | — | — | — | — |
| Donor 8 | — | — | — | — | — | 183 | — | — |
| Donor 9 | — | — | — | — | — | — | — | 268* |
| Donor 10 | — | — | — | — | — | — | 84 | 541 |
| Donor 11 | — | — | — | — | — | — | 329 | 52 |
| Donor 12 | — | — | — | — | — | — | 233 | — |

*PMBCs cultured in AIM medium
**PBMCs cultured in RPMI medium + group AB human serum

A significant secretion of IFN-γ could be demonstrated in 7 of the 12 donors. A secretion of IL-10 was also found in 4 of the 12 donors. No significant production of IL-4 or of IL-2 was found by ELISA. By comparison, the controls in the absence of peptide mixture were negative, with the exception of one case (*: IFN-γ found at 126 pg/ml).

1. An immunogenic peptide of the Epstein-Barr virus (EBV) comprising at least one CD4+ T epitope of one of the EBNA1, LMP1 or LMP2 latency antigens which can be presented by at least 7 different HLA II molecules chosen from the HLA-DR1, HLA-DR3, HLA-DR4, HLA-DR7, HLA-DR11, DLA-DR13, HLA-DR15, HLA-DRB3, HLA-DRB4, HLA-DRB5 and HLA-DP4 molecules, and in that it is selected from the group consisting of:
a) the peptides of, respectively, 26, 26 and 24 amino acids whose sequence extends from positions 475 to 500, 514 to 539, and 529 to 552 of EBNA1,
b) the peptide of 16 amino acids whose sequence extends from positions 68 to 83 of LMP1,
c) the peptides of, respectively, 21 and 17 amino acids whose sequence extends from positions 224 to 244 and 372 to 388 of LMP2, and
d) the variants of the same length and defined by the same positions as the peptides defined in a), b) and c), which variants are obtained by substitution of at least one amino acid of said peptides defined in a), b) and c) with another amino acid, and exhibit a binding activity of less than 1000 nM, with respect to at least 7 different HLA II molecules chosen from HLA-DR1, HLA-DR3, HLA-DR4, HLA-DR7, HLA-DR11, HLA-DR13, HLA-DR15, HLA-DRB3, HLA-DRB4, HLA-DRB5 and HLA-DP4.

2. The peptide as claimed in claim 1 wherein the HLA-II molecules comprise a β chain encoded, respectively, by the alleles DRB1*0101 (HLA-DR1 molecule), DRB1*0301 (HLA-DR3 molecule), DRB1*0401 (HLA-DR4 molecule), DRB1*0701. (HLA-DR7 molecule), DRB1*1101 (HLA-DR11 molecule), DRB1*1301 (HLA-DR13 molecule), DRB1*1501 (H LA-DR15 molecule), DRB3*0101 (HLA-DRB3 molecule), DRB5*0101 (HLA-DRB5 molecule), DP*0401 or DP*0402 (HLA-DP4 molecule).

3. The peptide as claimed in claim 1, wherein the peptide is selected from the group consisting of the sequences SEQ ID NOS: 4, 5, 6, 11, 20 and 21.

4. A polyepitope fragment comprising two identical or different epitopes, including at least one CD4+ T epitope whose sequence is that of the peptide as claimed in claim 1.

5. The polyepitope fragment as claimed in claim 4, comprising a CD4+ T epitope and at least one other epitope selected from the group consisting of:
a CD8+ T epitope of an EBV antigen,
a B epitope of an EBV antigen, and
a natural or synthetic universal CD4+ T epitope, such as the TT peptide 830-846, PADRE, the peptide 45-69 of the HIV-1 Nef protein and the CS.T3, CSP, SSP2, LSA-1 and EXP-1 peptides.

6. A fusion protein, wherein the fusion protein consists of a protein or a protein fragment, fused to a peptide as claimed in claim 1 or a polyepitope fragment thereof.

7. The fusion protein as claimed in claim 6, wherein the protein or the fragment is selected from the group consisting of an alpha or beta chain of an HLA II molecule chosen from the HLA-DR1, HLA-DR3, HLA-DR4, HLA-DR7, HLA-DR11, HLA-DR13, HLA-DR15, HLA-DRB3, HLA-DRB4, HLA-DRB5 and HLA-DP4 molecules or a fragment of said chain corresponding to a soluble HLA II molecule.

8. A lipopeptide comprising a lipid added to an α-amino functional or a reactive functional side chain of a peptide as claimed in claim 1 or of a polyepitope fragment thereof.

9. A polynucleotide encoding a peptide as claimed in claim 1 or a polyepitope fragment thereof.

10. An expression vector comprising a polynucleotide as claimed in claim 9, under the control of appropriate regulatory sequences for transcription and, optionally, translation.

11. A host cell comprising the expression vector as claimed in claim 10.

12. An immunogenic or vaccinal composition comprising at least one peptide, one polyepitope fragment, one lipopeptide or one expression vector as claimed in claim 1, and a pharmaceutically acceptable carrier and/or a carrier substance and/or an adjuvant.

13. The immunogenic composition as claimed in claim 12 comprising at least two of the EBV peptides as claimed in claim 1.

14. The immunogenic composition as claimed in claim 13, comprising at least one peptide including an epitope selected from the group consisting of an EBV CD8+ T epitope, an EBV B epitope and a universal CD4+ T epitope.

15. A vaccine comprising the peptide as claimed in claim 1 or a polyepitope fragment thereof for the prevention and/or treatment of an EBV infection or of an associated tumoral pathology.

16. A method for prevention and/or treatment of an EBV infection or of an associated tumoral pathology comprising administering the vaccine of claim 15 to an individual having an EBV infection or an associated tumoral pathology.

17. A reagent for diagnosing an EBV infection or an associated tumoral pathology comprising at least one peptide as claimed in claim 1, optionally labeled or complexed, in particular complexed with labeled HLA II molecules, in the form of multimeric complexes.

18. A method for diagnosing an EBV infection or an associated tumoral pathology comprising administering the peptide of claim 1 or a polyepitope fragment thereof to an individual.

19. A method of diagnosing an EBV infection or an associated tumoral pathology, comprising:
bringing a biological sample from an individual into contact with a diagnostic reagent as claimed in claim 17, and
detecting CD4+ T cells specific for one of the EBNA1, LMP1 or LMP2 latency antigens of EBV.

20. A method of sorting CD4+ T cells specific for one of the EBNA1, LMP1 or LMP2 latency antigens of EBV, comprising:
bringing a cell sample into contact with labeled multimeric complexes, formed by the binding of soluble HLA II molecules with at least one peptide as claimed in claim 1, and
sorting the cells bound to said HLA II/peptide complexes.
